(19) United States
(12) Patent Application Publication
(10) Pub. No.: US 2025/0002834 A1
(43) Pub. Date: Jan. 2, 2025

(54) SENSOR MEASUREMENT COMPENSATION IN BIOPROCESSING SYSTEMS

(71) Applicant: MERCK PATENT GMBH, Darmstadt (DE)

(72) Inventors: Laurent Jourdainne, Stutzheim-Offenhein (FR); Thomas Coton, Illkirch-Graffenstaden (FR); Anthony Da Silva, Bischoffshein (FR); Quentin Spindler, Colman (FR)

(21) Appl. No.: 18/709,715

(22) PCT Filed: Feb. 8, 2023
(86) PCT No.: PCT/EP2023/053032

§ 371 Date: May 13, 2024

(30) Foreign Application Priority Data
Feb. 11, 2022 (EP) 22305153.3

(51) Int. Cl. C12M 1/00; C12M 1/02; C12M 1/34; G01N 30/46; G01N 30/64

(52) U.S. Cl. CPC C12M 41/14; C12M 27/00; C12M 41/26; C12M 41/40; G01N 30/468; G01N 30/64

(57) ABSTRACT

A pH system that is able to compensate for variations in pressure and conductivity is disclosed. The system includes a pH sensor, a pressure sensor and optionally a conductivity sensor, the outputs of each are transmitted to a controller. The controller multiplies a compensation coefficient by the difference in pressure between the measured pressure and a threshold pressure. This result is then added to the measured pH value. Further, the compensation coefficient is a function of the conductivity of the solution. In some instances, the compensation coefficient is calculated by raising the conductivity to a power and multiplying that result by a constant. This pH system may be incorporated into various bioprocessing systems, such as multi-column chromatography systems and viral inactivation systems.

### FIELD

Embodiments of the present disclosure relate to systems and method to compensate the measurements received from sensors based on other environmental conditions.

### BACKGROUND

In many applications, such as bioprocessing applications, it is important to carefully and accurately monitor and control parameters associated with the environment, such as pH, pressure, temperature, conductivity and others. These parameters may determine the efficacy of the particular process, and may be critical to the desired result.

For example, in viral inactivation applications, pH must be tightly controlled in order to ensure that the virus is inactivated without degrading the product quality. This is also true for various other processes.

Further, in some bioprocessing applications, the various operating parameters at various points in the system are displayed for the user. Accuracy in these displayed parameters is paramount to instilling trust in the user.

Therefore, it would be beneficial if there was a system that produced accurate pH sensor readings, which have been adjusted based on pressure and conductivity. Further, it would be advantageous if these adjusted or compensated values were utilized in bioprocessing applications.

### SUMMARY

A pH system that is able to compensate for variations in pressure and conductivity is disclosed. The system includes a pH sensor, a pressure sensor and optionally a conductivity sensor, the outputs of each are transmitted to a controller. The controller multiplies a compensation coefficient by the difference in pressure between the measured pressure and a threshold pressure. This result is then added to the measured pH value. Further, the compensation coefficient is a function of the conductivity of the solution. In some instances, the compensation coefficient is calculated by raising the conductivity to a power and multiplying that result by a constant. This pH system may be incorporated into various bioprocessing systems, such as multi-column chromatography systems and viral inactivation systems.

### BRIEF DESCRIPTION OF THE FIGURES

For a better understanding of the present disclosure, reference is made to the accompanying drawings, which are incorporated herein by reference and in which:

FIG. 1 is a representative graph showing the effect of pressure on pH sensor measurements for various selected solution conductivities;

FIG. 2 is a representative graph showing the effect of conductivity on the pressure compensation coefficient;

FIGS. 3-5 illustrate a multi-column chromatography system in different configurations;

FIG. 6 illustrates a viral inactivation system; and

FIG. 7 shows a system having multiple sensors and a controller.

### DETAILED DESCRIPTION

Embodiments of the present disclosure describe the system and method to compensate for inaccuracies in sensor measurement due to environmental conditions.

In many applications, such as bioprocessing applications, it is important to carefully and accurately monitor and control parameters associated with the environment, such as pH, pressure, temperature, conductivity and others.

It is known that temperature may affect the accuracy of a pH sensor. In fact, many pH sensors are designed with integrated temperature sensors to allow compensation for temperature variation.

However, unexpectedly, it has been found that pH sensors are also sensitive to other parameters.

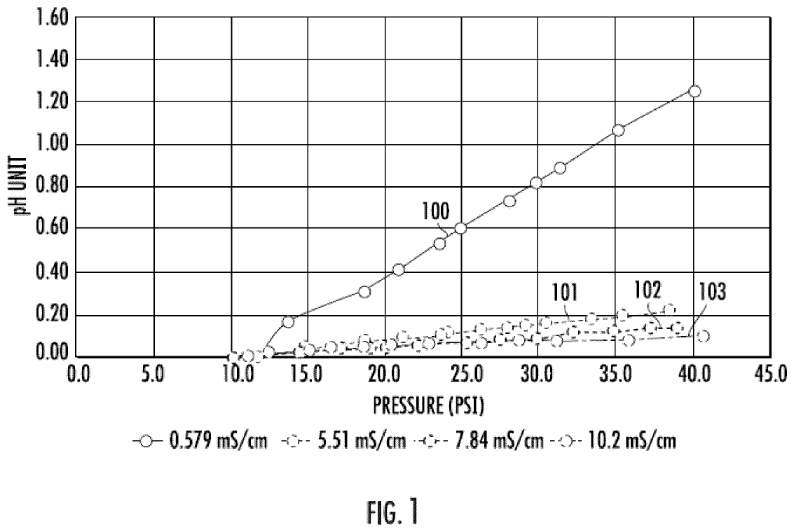

FIG. 1 shows a graph shows the pH reading for a sensor in a fluid having a known pH while pressure and conductivity are being varied. Line 100 shows the pH measurements as a function of pressure for a solution of a known pH having a conductivity of 0.579 mS/cm. As can be seen, the pH increases linearly with increasing pressure at a first slope. Line 101 shows the pH measurements as a function of pressure for a solution of a known pH having a conductivity of 5.51 mS/cm. As can be seen, the pH increases linearly with increasing pressure at a second slope, which is less than the first slope. Line 102 shows the pH measurements as a function of pressure for a solution of a known pH having a conductivity of 7.84 mS/cm. As can be seen, the pH increases linearly with increasing pressure at a third slope, which is less than the second slope. Finally, line 103 shows the pH measurements as a function of pressure for a solution of a known pH having a conductivity of 10.2 mS/cm. As can be seen, the pH increases linearly with increasing pressure at a fourth slope, which is smaller than the other slopes.

Note that for each line, the slopes are approximately linear. In other words, pH seems to vary linearly as a function of pressure for all pressures greater than a threshold pressure. The slope of each line is related to the conductivity of the solution. Stated differently, whenever P_{measured}>P_{threshold},

$$p {H}_{actual} = p {H}_{measured} + compensation\_coefficient ( σ ) * ( {P}_{measured} - {P}_{threshold} )$$

### Where:

P_{measured} is the pressure of the environment;
P_{threshold} is the threshold pressure, which may be 10 psi

Compensation_coefficient (σ) is the slope of the line as a function of pressure, where the slope is a function of conductivity (σ).

Below this pressure, no compensation is needed.

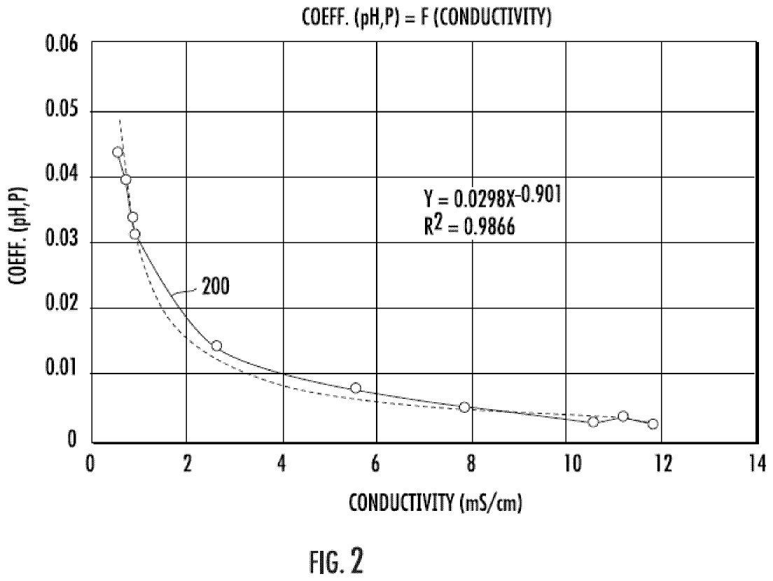

Using FIG. 1, the slope at each of the four conductivity values can be determined. FIG. 2 shows the relationship between these slope values and conductivity, which is represented as line 200.

As can be seen, there is a power law relationship between conductivity and the slope of the pressure line. In one embodiment, this relationship can be approximated as:

compensation_coefficient (σ)=0.0298σ^{-0.901}

In other words, the higher the conductivity of the solution, the less the pH measurement is affected by pressure. Thus, in certain embodiments, the compensation coefficient may be calculated by raising the conductivity value to a power, and then multiplying the result by a constant. In some embodiments, the power may be a negative number.

Combining these results yields:

$$p {H}_{actual} ( {P}_{measured} , σ ) = p {H}_{measured} + 0.0298 {σ}^{- 0.901} * ( {P}_{measured} - {P}_{threshold} )$$

Note that the above relationship is one specific embodiment. Rather, the generic form of this equation is expressed as:

$$p {H}_{actual} ( {P}_{measured} , σ ) = {pH}_{measured} + compensation\_coefficient ( σ ) * ( {P}_{measured} - {P}_{threshold} )$$

This compensation is important, since the range of pressure within bioprocessing equipment is typically in the range of 0 to 4 bars (58 psi), and the conductivity of the solution, which may be either a buffer or biomaterial solution, may be between 500 μS/cm an several tens of mS/cm. This wide variation in operating conditions may lead to pH readings that may be inaccurate.

Having determined that the actual pH of a solution is actually a function of pressure and conductivity, this knowledge can be used to improve the operation of various bioprocesses.

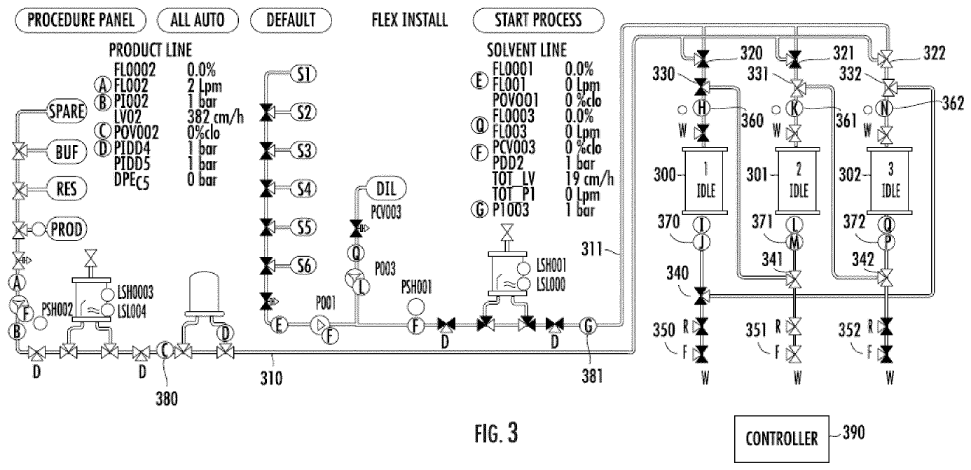

FIGS. 3-5 show a multi-column chromatography system, having three columns; first column 300, second column 301 and third column 302. A controller 390 is used with this system, and is in communication with the valves, the pressure sensors, the pH sensors and the conductivity sensors.

A product fluid conduit 310 is used to deliver product materials to one or more of the columns. For example, the product fluid conduit 310 may deliver one or more of the following: buffer, product, and other materials. Various mixing valves are used to introduce these components to the product fluid conduit 310.

A solvent fluid conduit 311 is used to deliver one or more solvents to one or more of the columns. Various mixing valves are used to introduce one or more of a plurality of different solvents to the solvent fluid conduit 311.

An inlet mixing valve is associated with each column. First inlet mixing valve 320 is disposed in a position to enable or disable the flow of product and/or solvent into the first column 300. Second inlet mixing valve 321 is disposed in a position to enable or disable the flow of product and/or solvent into the second column 301. Third inlet mixing valve 322 is disposed in a position to enable or disable the flow of product and/or solvent into the third column 302.

An input serial mixing valve is associated with each column. First input serial mixing valve 330 is disposed in a position to enable or disable the flow of material from the output of the second column 301 into the first column 300. Second input serial mixing valve 331 is disposed in a position to enable or disable the flow of material from the output of the third column 302 into the second column 301. Third input serial mixing valve 332 is disposed in a position to enable or disable the flow of material from the output of the first column 300 into the third column 302.

Additionally, an output serial mixing valve is associated with each column. First output serial mixing valve 340 is disposed in a position to enable or disable the flow of material from the output of the first column 300 into the third column 302. Second output serial mixing valve 341 is disposed in a position to enable or disable the flow of material from the output of the second column 301 into the first column 300. Third output serial mixing valve 342 is disposed in a position to enable or disable the flow of material from the output of the third column 302 into the second column 302.

Each output serial mixing valve is also in communication with a respective outlet valve 350, 351, 352, which, when open, drains the material from one or more columns to waste, to a storage tank or to another system.

Note that the columns and mixing valves may be configured differently. For example, the output from the third column 302 may be fed to the first column 300; the output from the first column 300 may be fed to the input of the second column 301 and the output of the second column 301 may be fed to the input of the third column 302. Thus, FIGS. 3-5 are illustrating one specific embodiment; however, the disclosure is not limited to this embodiment.

Sensors may be located at various points within this multi-column chromatography system. For example, pH sensors and conductivity sensors may be disposed at locations 360, 361 and 362. Pressure sensors may be disposed at locations 370, 371 and 372. Additionally, pressure sensors may be disposed at locations 380 and 381.

In FIG. 3, the third column 302 and the second column 301 are being loaded. To do this, product flows through product fluid conduit 310 and though third input mixing valve 322, which is open. Note that second input mixing valve 321 is closed at this time. The product flows through third input serial mixing valve 332 and into the third column 302. Product then flows out from the output of the third column 302 and is routed by output serial mixing valve 342 into the second input serial mixing valve 331 and into the input of the second column 301. The product then flows through the second column 301 and exits through second output serial mixing valve 341 and second outlet valve 351. The remaining valves are closed. Thus, in this configuration, it may be beneficial to measure the pH in the third column 302 and the second column 301. As noted above, pH sensors may be disposed at locations 360, 361, and 362. Additionally, conductivity sensors may also be disposed in these locations.

While the third column 302 and the second column 301 are being loaded, the first column 300 is being unloaded. This is done by allowing solvent to pass through solvent fluid conduit 311 and into the first input mixing value 320. The solvent passes through the first column 300 and exits through first output serial mixing valve 340.

Further, during the loading process, the pressure in the third column 302 may be approximated using the pressure sensor disposed at location 380. The conductivity of the material entering the third column 302 may be measured using the conductivity sensor disposed at location 362. The pressure in the second column 301 may be approximated using the pressure sensor disposed at the output of the third column 302, at location 372. The conductivity of the material entering the second column 302 may be measured using the conductivity sensor disposed at location 361. Using the conductivity measurements and the pressure measurements from these sensors, the reading from the pH sensors disposed in locations 361 and 362 may be compensated.

In another embodiment, pressure sensors may be located at locations 360, 361 and 362.

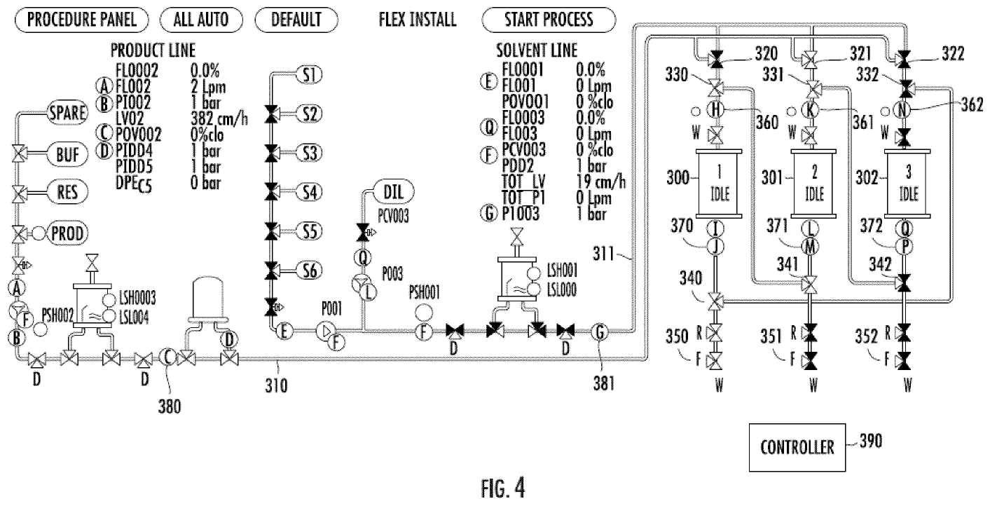

FIG. 4 shows the loading of the second column 301 and the first column 300. To do this, product flows through product fluid conduit 310 and though second input mixing valve 321, which is open. Note that first input mixing valve 320 is closed at this time. The product flows through second input serial mixing valve 331 and into the second column 301. Product then flows out from the output of the second column 301 and is routed by output serial mixing valve 341 into the first input serial mixing valve 330 and into the input of the first column 300. The product then flows through the first column 300 and exits through first output serial mixing valve 340 and first outlet valve 351. The remaining valves are closed. Thus, in this configuration, it may be beneficial to measure the pH in the second column 301 and the first column 300.

While the second column 301 and the first column 300 are being loaded, the third column 302 is being unloaded. This is done by allowing solvent to pass through solvent fluid conduit 311 and into the third input mixing value 322. The solvent passes through the third column 302 and exits through third output serial mixing valve 342.

As noted above, pH sensors may be disposed at locations 360, 361, and 362. Additionally, conductivity sensors may also be disposed in these locations. Further, the pressure in the second column 301 may be approximated using the pressure sensor disposed at location 380. The pressure in the first column 301 may be approximated using the pressure sensor disposed at the output of the second column 301, at location 371. Using these measurements, the pH readings for the second column 301 and the first column 300 may be compensated.

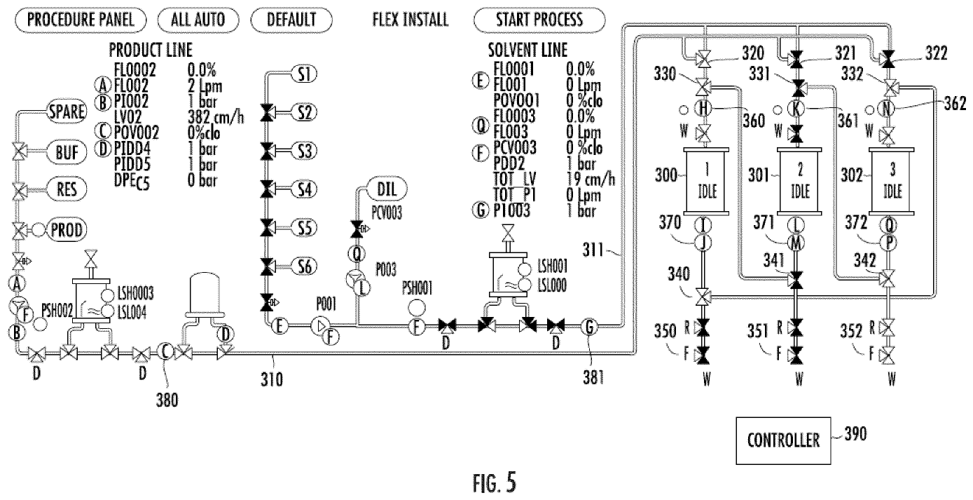

FIG. 5 shows the loading of the first column 300 and the third column 302. To do this, product flows through product fluid conduit 310 and though first input mixing valve 320, which is open. Note that third input mixing valve 322 is closed at this time. The product flows through first input serial mixing valve 330 and into the first column 300. Product then flows out from the output of the first column 300 and is routed by first output serial mixing valve 340 into the third input serial mixing valve 332 and the input of the third column 302. The product then flows through the third column 302 and exits through third output serial mixing valve 342 and third outlet valve 352. The remaining valves are closed. Thus, in this configuration, it may be beneficial to measure the pH in the first column 300 and the third column 302.

While the first column 300 and the third column 302 are being loaded, the second column 301 is being unloaded. This is done by allowing solvent to pass through solvent fluid conduit 311 and into the second input mixing value 321. The solvent passes through the second column 301 and exits through second output serial mixing valve 341.

As noted above, pH sensors may be disposed at locations 360, 361, and 362. Additionally, conductivity sensors may also be disposed in these locations. Further, the pressure in the first column 300 may be approximated using the pressure sensor disposed at location 380. The pressure in the third column 301 may be approximated using the pressure sensor disposed at the output of the first column 300, at location 370. Using these measurements, the pH readings for the first column 300 and the third column 302 may be compensated.

In addition, various sensors may be used during the unloading and regeneration process. The pressure sensor disposed at location 381 may be used to indicate the pressure entering a column during the unloading process. In FIG. 3, the first column 300 is being unloaded. The conductivity sensor and pH sensor disposed at location 360 may be used to compensate for the pressure reading, if necessary. In FIG. 4, the third column 302 is being unloaded. The conductivity sensor and pH sensor disposed at location 362 may be used to compensate for the pressure reading, if necessary. In FIG. 5, the second column 301 is being unloaded. The conductivity sensor and pH sensor disposed at location 361 may be used to compensate for the pressure reading, if necessary.

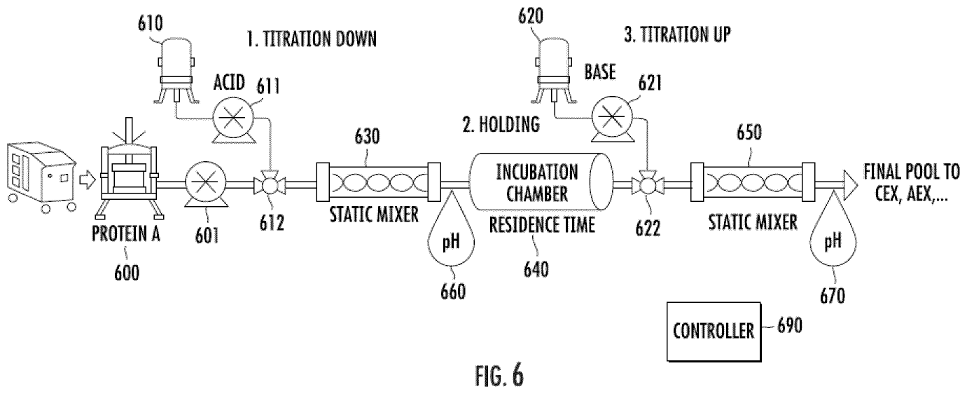

FIG. 6 shows another embodiment where the compensated pH sensor may be utilized. In this figure, a system for viral inactivation is shown. The system for viral inactivation includes a controller 690. The controller 690 is in communication with the valves, pumps, pressure sensors, pH sensors and conductivity sensors.

In operation, a protein 600 may be delivered using protein pump 601. The protein 600 passes through first mixing valve 612. Additionally, an acid 610 is delivered to the first mixing valve 612 using acid pump 611. The protein 600 and acid 610 then enter a first static mixer 630, which is downstream from the first mixing valve 612, where they are thoroughly mixed to form a mixture. The mixture then enters the incubation chamber 640, where it is allowed to dwell for a predetermined amount of time. Upon exiting the incubation chamber 640, the mixture passes through a second mixing valve 622. Additionally, a base 620 is delivered to the second mixing valve 622 using base pump 621. The mixture and the base 620 then enter a second static mixer 650, which is downstream from the second mixing valve 622, where they are thoroughly mixed. The output of the static mixer 650 may then be used as the input to an anion exchange (AEX), a cation exchange (CEX) or other device or system.

It may be beneficial to measure the pH of the mixture exiting the first static mixer 630, such as at location 660. Therefore, a first pH sensor may be installed in this location. However, to compensate for variations in pressure and conductivity, a first conductivity sensor and/or a first pressure sensor may also be disposed at location 660. This allows a first compensated pH value of the mixture of the acid and the protein to be computed by the controller 690.

However, in certain embodiments, the conductivity of the mixture may not vary significantly. In this embodiment, the conductivity sensor may be disposed in another location, such as before the first static mixer 630, before the first mixing valve 612, or even at the source of the protein 600. In other words, the conductivity sensor may be disposed upstream of the incubation chamber 640.

In other embodiments, the conductivity of the mixture may be known and may be manually input to the controller 690. In this case, a conductivity sensor may not be needed.

Similarly, it may be beneficial to measure the pH of the mixture exiting the second static mixer 650, such as at location 670. Therefore, a second pH sensor may be installed in this location. However, to compensate for variations in pressure and conductivity, a second pressure sensor may also be disposed at location 670. In some embodiments, a second conductivity sensor is also used. This allows a second compensated pH value of the mixture of the base, acid and the protein to be computed by the controller 690. In other embodiments, the conductivity provided earlier is suitable for use in determining the second compensated pH value and therefore, a second conductivity sensor is not used.

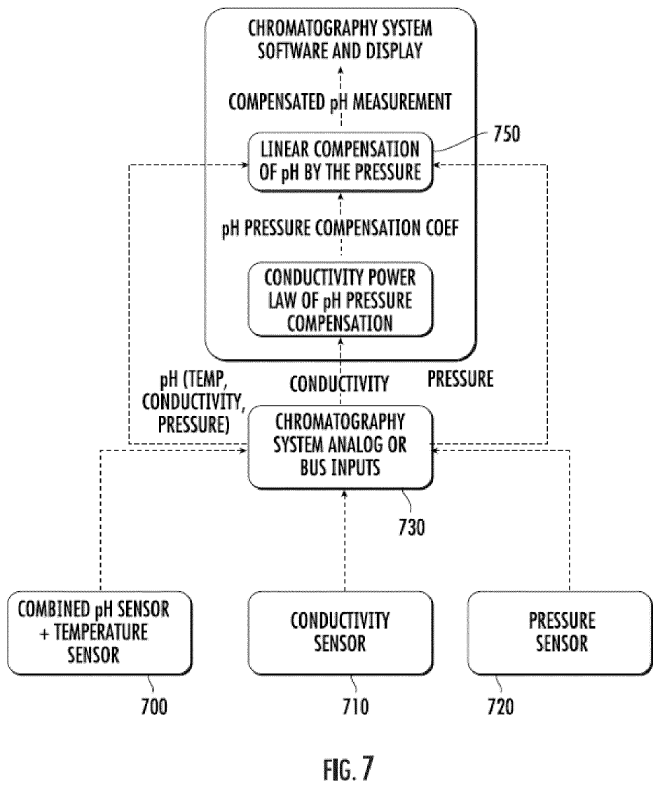

FIG. 7 shows an example embodiment that is applicable to either of the bioprocessing systems described above. Typically, a pH sensor, such as pH sensor 700 includes an integrated temperature sensor to correct for known temperature dependence. Each of the bioprocessing systems described herein may also have a conductivity sensor 710. In certain embodiments, where the conductivity of the solution is known, a conductivity sensor may not be used. Rather, the conductivity of the solution may be provided to the controller 750 in another manner. The conductivity may be manually entered or may be inferred from the buffer being utilized. Additionally, each of the bioprocessing systems described herein may also have at least one pressure sensor 720. Measurements from each of these sensors may be transmitted via a communications system 730, which may be analog signals or a bus.

The three values; conductivity, pressure and pH are then supplied to the controller 750. The controller 750 first uses the conductivity to determine the slope of the pH/pressure graph, also referred to as the compensation coefficient. As described above, the relationship between the compensation coefficient and the conductivity may be a power law, where the conductivity is raised to a power and then multiplied by a constant to yield the slope. The power may be a negative number.

Once the compensation coefficient has been determined, this value is then used to compensate for the pressure of the solution. As described above, at pressures above a threshold pressure, the relationship between pressure and pH is linear, where the slope is determined based on the conductivity of the solution. Thus, the compensation coefficient is multiplied by the difference between the measured pressure and the threshold pressure, if the measured pressure is greater than the threshold pressure. This result is then added to the measured pH value to yield a compensated value, which is much more accurate than the measured pH value. This compensated pH value may then be used by the controller 750 in a plurality of ways. In one embodiment, the value may be displayed to a user. As another example, the value may be used by the controller 750 to control the operation of the system. As noted above, if the measured pressure is less than the threshold pressure, no compensation is required.

As described above, in some embodiments, the pressure sensor and the conductivity sensor may be collocated with the pH sensor so that all relevant measurements are taken from the same physical location. In other embodiments, the conductivity of the solution may be constant and a known value, such that a conductivity sensor is not needed; rather, the conductivity is simply entered into the controller. In another embodiment, the pressure sensor may not be collocated with the pH sensor, but may be disposed in a location that has the same pressure as the location where the pH sensor is disposed.

The embodiments described above in the present application may have many advantages. For example, with respect to the multi-column chromatography system in FIGS. 3-5, the pressure at the input to the first column being loaded is much greater than the pressure in the second column being loaded. Using conventional pH sensors, a user of this system may be confused or dissatisfied that the pH readings in these two columns differ by up to 0.4. By using conductivity and pressure to compensate for the pH sensors, the readings from the two columns are much closer together. In one test, the compensated pH readings were within 0.03, which is less than the tolerance of the pH sensors. With respect to the viral inactivation, the actual pH value is critical as it must stay between an upper and lower limit. If the actual pH is too low, the product quality may deteriorate and the production yield may be reduced. If the actual pH value is too high, the inactivation may fail and the viruses may remain in the solution, giving rise to safety risks, since it was believed that the inactivation was successful.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A system for measuring pH, comprising:
a pH sensor to measure a pH of a solution;
a pressure sensor to measure a pressure; and
a controller, wherein the controller is in communication with the pH sensor and the pressure sensor, and wherein the controller computes a compensation amount by multiplying a compensation coefficient by a difference between the pressure as measured by the pressure sensor and a threshold pressure, and adds the compensation result to the pH measured by the pH sensor to obtain a compensated pH.

2. The system of claim 1, wherein the compensation coefficient is a function of a conductivity of the solution.

3. The system of claim 2, further comprising a conductivity sensor to measure the conductivity of the solution.

4. The system of claim 2, wherein the conductivity of the solution is supplied to the controller.

5. The system of claim 2, wherein the compensation coefficient is the conductivity of the solution, raised to a power and multiplied by a constant.

6. The system of claim 5, wherein the power is a negative number.

7. The system of claim 3, wherein the conductivity sensor, pressure sensor and the pH sensor are collocated.

8. The system of claim 3, wherein the conductivity sensor and the pH sensor are collocated and the pressure sensor is disposed in a location that has the same pressure as a location where the pH sensor is disposed.

9. A multi-column chromatography system, comprising:
at least three columns;
a plurality of valves to direct flow to and from the at least three columns;
a pressure sensor;
a conductivity sensor;
a pressure sensor; and
a controller, wherein the controller receives inputs from the pressure sensor, the conductivity sensor and the pH sensor and determines a compensated pH value.

10. The multi-column chromatography system of claim 9, wherein the controller computes a compensation amount by multiplying a compensation coefficient by a difference between a pressure as measured by the pressure sensor and a threshold pressure, wherein the compensation coefficient is a function of a conductivity measured by the conductivity sensor; and adds the compensation result to the pH measured by the pH sensor to obtain the compensated pH.

11. A system for viral inactivation, comprising:
a source of a protein;
a source of acid;
a source of base;
a first mixing valve in communication with the source of protein and the source of acid;
a first static mixer downstream from the first mixing valve wherein an output of the first static mixer comprises a mixture;
an incubation chamber in communication with an output of the first static mixer;
a first pH sensor disposed between the output of the first static mixer and the incubation chamber;
a first pressure sensor to measure a pressure of the mixture; and
a controller, wherein the controller receives information about a conductivity of the mixture and receives inputs from the first pressure sensor and the first pH sensor and determines a first compensated pH value.

12. The system of claim 11, wherein the first conductivity sensor is disposed between the output of the first static mixer and the incubation chamber.

13. The system of claim 11, wherein a conductivity sensor is disposed upstream from the incubation chamber to provide the information about the conductivity of the mixture.

14. The system of claim 11, wherein the information about the conductivity of the mixture is manually supplied to the controller.

15. The system of claim 11, further comprising:
a second mixing valve in communication with an output of the incubation chamber and the source of base;
a second static mixer downstream from the second mixing valve;
a second pH sensor disposed at the output of the second static mixer; and
a second pressure sensor disposed at the output of the second static mixer; wherein the controller receives information about the conductivity of the mixture and inputs from the second pressure sensor and the second pH sensor and determines a second compensated pH value.
